(19) United States
(12) Patent Application Publication
(10) Pub. No.: US 2002/0172615 A1
(43) Pub. Date: Nov. 21, 2002

(54) Apparatus for and method of manufacturing a prefilled sterile container

(76) Inventors: Archie Woodworth, Barrington, IL (US); John Falzone, Crystal Lake, IL (US); John Darvasi, Hawthorn Wood, IL (US); Amy N. Gillum, Lake Villa, IL (US); James Kamienski, Chicago, IL (US); Margaret J. Barnato, Lake Forest, IL (US); Robert Gliniecki, Spring Grove, IL (US)

Correspondence Address: Wallenstein & Wagner, Ltd., 311 S. Wacker Drive, 53rd Floor, Chicago, IL, 60606-6630, US

(21) Appl. No.: 09/802,315

(22) Filed: Mar. 8, 2001

(51) Int. Cl. A61L 2/00

(52) U.S. Cl. 422/22

(57) ABSTRACT

The present invention claims a method of producing sterile prefilled syringe bodies. The method comprises the steps of providing a syringe body. The syringe bodies are sterilized and transferred to a sterile environment. While the syringe bodies are maintained in a sterilized condition as they are transferred to the sterile environment. A fluid substance is provided and introduced into the syringe body while the syringe body is within the sterile environment. The fluid substance is also sealed within the syringe body while the syringe body is within the sterile environment.

### DESCRIPTION

1. Technical Field

The present invention relates generally to an apparatus for and method of producing sterile polymeric containers, and more specifically to an apparatus for and method of continuous production of sterile, prefilled polymeric syringe bodies.

2. Background Prior Art

Typically, glass syringe bodies are manufactured by producing the syringe body in a production plant. The syringe bodies are packaged and shipped to a pharmaceutical plant where they are unpackaged, filled, sealed tightly, and sterilized. The syringe bodies are then repackaged and ready to be delivered to the end user. This process is inefficient and costly.

Recently, syringe bodies have been manufactured from polymeric resins. The polymeric syringe bodies replaced glass syringe bodies which were costly to produce and caused difficulties during the manufacturing process because the glass would chip, crack, or break. The broken glass particles would not only become hazards to workers and manufacturing equipment, but would also become sealed within the glass syringe body causing a potential health hazard to a downstream patient.

U.S. Pat. No. 6,065,270 (the '270 patent), issued to Reinhard et al. and assigned to Schott Glaswerke of Germany, describes a method of producing a prefilled, sterile syringe body from a cyclic olefin copolymer (COC) resin. A COC polymer is useful in the manufacture of syringe bodies because it is generally clear and transparent. COC resins are, for example, disclosed in U.S. Pat. No. 5,610,253 which is issued to Hatke et al. and assigned to Hoechst Akteiengesellschaft of Germany.

The '270 patent includes a method of manufacturing a filled plastic syringe body for medical purposes. The syringe body comprises a barrel having a rear end which is open and an outlet end with a head molded thereon and designed to accommodate an injection element, a plunger stopper for insertion into the rear end of the barrel to seal it, and an element for sealing the head. The method of manufacturing the syringe body includes the steps of: (1) forming the syringe body by injection molding a material into a core in a cavity of an injection mold, the mold having shape and preset inside dimensions; (2) opening and mold and removing the formed syringe body, said body having an initial temperature; (3) sealing one end of the barrel of the plastic syringe body; (4) siliconizing an inside wall surface of the barrel of the plastic syringe body immediately after the body is formed and while the body remains substantially at said initial temperature; (5) filling the plastic syringe body through the other end of the barrel of the plastic syringe body; and (6) sealing the other end of the barrel of the plastic syringe body, wherein the method is carried out in a controlled environment within a single continuous manufacturing line. According to the method of the '270 patent, the sterilization step is applied to the filled and completely sealed ready-to-use syringe body. Historically, sterilization of finished syringe components (barrel, plunger, and tip cap) has been conducted using ethylene oxide, moist-heat or gamma irradiation.

### SUMMARY OF THE INVENTION

An object of the present invention is to provide a process by which sterile prefilled syringe bodies for medical applications are continuously produced. The syringe bodies are of the type having at least one interior chamber defined by an inner cylindrical sidewall, a tip end having an opening adapted for receiving an injection needle or the like, and a larger open end for receiving a plunger for activating a flow of a fluid substance outwardly from the chamber through the tip end. Such syringe bodies are commonly used in medical applications.

The process of the present invention generally comprises the steps of sterilizing empty molded syringe bodies, transferring the syringe bodies to a sterilization station, sterilizing the syringe bodies, transferring the syringe bodies to a sterile environment, and processing the syringe bodies within the sterile environment to produce a prefilled, sterile syringe body. The process may also include the following steps: producing a plurality of syringe bodies, transferring the syringe bodies to a packaging station, and packaging the syringe bodies.

The process begins with the producing the syringe bodies step. The producing the syringe bodies step includes continuously producing a plurality of syringe bodies. Once the syringe bodies are molded, each syringe body is transferred to a quality control station where each one is inspected and weighed. Syringe bodies which satisfy a predetermined specification are transferred to a tip cap station where tip caps are added to each syringe body to effectively seal and close the tip end of the syringe body. Next, the interior of the syringe bodies are lubricated, preferably with silicone.

During the transferring the syringe bodies to a sterilization station step, the syringe bodies are transported along a conveyor to a sterilization station. The sterilization station may include a terminal process performed within an autoclave or an irradiation process.

Once the syringe bodies are sterilized, they are sterile transferred to a sterile environment. The sterile environment is typically an enclosed isolator or other sterile environment. Each syringe body enters the sterilization station and remains unwrapped and sterilized.

Next, the syringe bodies are processed within the sterile environment. The process includes the steps of filling each syringe body with a sterile medical solution, transferring a sterile plunger for each syringe body into the sterile environment, and adding a plunger to an open end of each syringe body. The medical solution is generally introduced into the syringe bodies via the open end of the syringe body which is opposite the tip capped end.

The plungers are sterilized prior to being sterile transferred into the isolator and may be sterilized in any conventional manner. Once a syringe body is filled with the medical solution, a plunger is inserted into the open end of the syringe body. Once inserted within the open end of the syringe body, the plunger forms a seal with an inner sidewall of the syringe body wherein the medical solution is sealed within the syringe body.

The next step is transferring the syringe body to the packaging station from the isolator. Syringe bodies are typically transferred along conveyor; however, any transfer mechanism can be used.

This transfer step includes the step of transferring the syringe bodies from the isolator and may optionally include a post-fill sterilization step. In this optional sterilization step, the syringe bodies and the contents thereof are sterilized either by steam or ultraviolet radiation.

Following the optional post-fill sterilization step, the syringe bodies are transferred from the optional sterilization station to the packaging station. During the packaging station step, a plunger rod is fixedly attached to each plunger, and the finished syringes are inspected, labeled, and packaged for shipment to an end user.

Other features and advantages of the invention will be apparent from the following specification taken in conjunction with the following drawings.

### BRIEF DESCRIPTION OF THE DRAWINGS

FIG. 1 is a view of a syringe body;

FIG. 2 is a flowchart of the method of the present invention;

FIG. 3 is a flowchart of a second embodiment of the method of the present invention;

FIG. 4 is a flowchart of a third embodiment of the method of the present invention; and

FIG. 5 is a plot showing the trend in pH of the sterile water for injection within a syringe of the present invention days to fill.

### DETAILED DESCRIPTION

While this invention is susceptible of embodiments in many different forms, there are shown in the drawings and will herein be described in detail, preferred embodiments of the invention with the understanding that the present disclosures are to be considered as exemplifications of the principles of the invention and are not intended to limit the broad aspects of the invention to the embodiments illustrated.

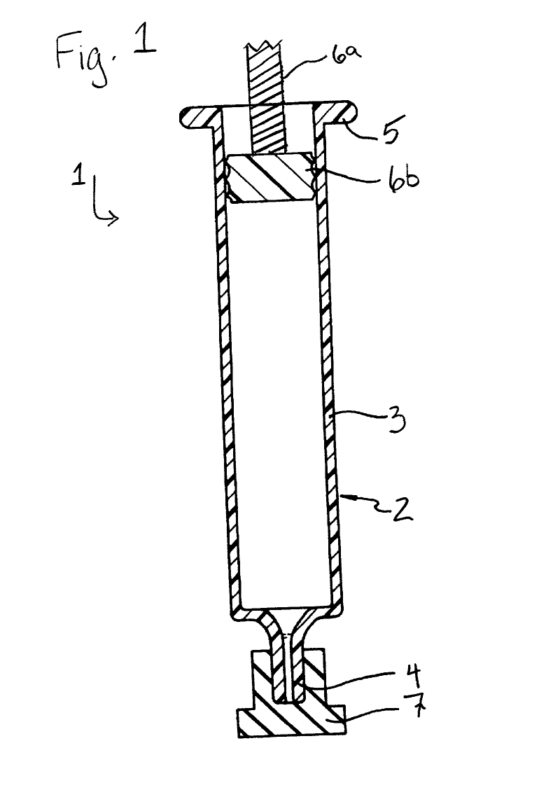

The present invention is directed to a method for continuously producing sterile prefilled container, such as a medical vial but preferably a prefilled, sterile, polymeric syringe body. Throughout this specification, syringe bodies are used as an illustrative example of the type of container provided; however, it should be understood that method of the present invention can be applied to any containers, vials, other types of storage vessels, or IV kits without departing from the spirit of the invention. Referring to FIG. 1, the syringe bodies 1 are of the type having at least one interior chamber 2 defined by an inner cylindrical sidewall 3, a tip end 4 having an opening adapted for receiving an injection needle or the like and a larger open end 5 for receiving a plunger arm 6a having a plunger seal 6b at a distal end of the plunger arm for activating a flow of a fluid substance outwardly from the chamber 2 through the tip end 4. The tip ends 4 are typically equipped with a tip cap 7. Such syringe bodies 1 are commonly used in medical applications.

I. Syringe Bodies

The syringe bodies 1 can be produced from glass or any suitable polymer, but are preferably produced from cyclic olefin containing polymers or bridged polycyclic hydrocarbon containing polymers. These polymers, in some instances, shall be collectively referred to as COCs.

The use of COC-based syringe bodies overcome many of the drawbacks associated with the use of glass syringe bodies. The biggest drawbacks of glass syringe bodies are in connection with the handling of the glass syringes. For instance, the glass syringes are often chipped, cracked, or broken during the manufacturing process. Glass particles may become trapped within the syringe bodies and subsequently sealed within the syringe barrel with the medical solution. This could be hazardous to a patient injected with the medical solution. Additionally, the glass particles could become a manufacturing hazard by causing injury to plant personnel or damage to expensive manufacturing equipment.

Suitable COC polymers include homopolymers, copolymers and terpolymers. obtained from cyclic olefin monomers and/or bridged polycyclic hydrocarbons as defined below.

Suitable cyclic olefin monomers are monocyclic compounds having from 5 to about 10 carbons in the ring. The cyclic olefins can be selected from the group consisting of substituted and unsubstituted cyclopentene, cyclopentadiene, cyclohexene, cyclohexadiene, cycloheptene, cycloheptadiene, cyclooctene, cyclooctadiene. Suitable substituents include lower alkyl, acrylate derivatives and the like.

Suitable bridged polycyclic hydrocarbon monomers have two or more rings and more preferably contain at least 7 carbons. The rings can be substituted or unsubstituted. Suitable substitutes include lower alkyl, aryl, aralkyl, vinyl, allyloxy, (meth) acryloxy and the like. The bridged polycyclic hydrocarbons are selected from the group consisting of those disclosed in the below incorporated patents and patent applications and in a most preferred form of the invention is norbornene.

Suitable homopolymer and copolymers of cyclic olefins and bridged polycyclic hydrocarbons and blends thereof can be found in U.S. Pat. Nos. 5,218,049, 5,854,349, 5,863,986, 5,795,945, 5,792,824; EP 0 291,208, EP 0 283,164, EP 0 497,567 which are incorporated in their entirety herein by reference and made a part hereof. These homopolymers, copolymers and polymer blends may have a glass transition temperature of greater than 50° C., more preferably from about 70° C. to about 180° C., a density greater than 0.910 g/cc and more preferably from 0.910 g/cc to about 1.3 g/cc and most preferably from 0.980 g/cc to about 1.3 g/cc and have from at least about 20 mole % of a cyclic aliphatic or a bridged polycyclic in the backbone of the polymer more preferably from about 30-65 mole % and most preferably from about 30-60 mole %.

Suitable comonomers for copolymers and terpolymers of the COCs include α-olefins having from 2-10 carbons, aromatic hydrocarbons, other cyclic olefins and bridged polycyclic hydrocarbons.

The presently preferred COC is a norbomene and ethylene copolymer. These norbornene copolymers are described in detail in U.S. Pat. Nos. 5,783,273, 5,744,664, 5,854,349, and 5,863,986. The norborene ethylene copolymers preferably have from at least about 20 mole percent norbornene monomer and more preferably from about 20 mole percent to about 75 mole percent and most preferably from about 30 mole percent to about 60 mole percent norbornene monomer or any combination or subcombination of ranges therein. The norbornene ethylene copolymer should have a glass transition temperature of from about 70-180° C., more preferably from 70-130° C. The heat deflection temperature at 0.45 Mpa should be from about 70° C. to about 200° C., more preferably from about 75° C. to about 150° C. and most preferably from about 76° C. to about 149° C. Also, in a preferred form of the invention, the COC is capable of withstanding, without significant heat distortion, sterilization by an autoclave process at 121° C. Suitable copolymers are sold by Ticona under the trade,name TOPAS under grades 6013, 6015 and 8007 (not autoclavable).

Other suitable COCs are sold by Nippon Zeon under the tradename ZEONEX and ZEONOR, by Daikyo Gomu Seiko under the tradeanme CZ resin, and by Mitsui Petrochemical Company under the tradename APEL.

It may also be desirable to have pendant groups associated with the COCs. The pendant groups are for compatibilizing the COCs with more polar polymers including amine, amide, imide, ester, carboxylic acid and other polar functional groups. Suitable pendant groups include aromatic hydrocarbons, carbon dioxide, monoethylenically unsaturated hydrocarbons, acrylonitriles, vinyl ethers, vinyl esters, vinylamides, vinyl ketones, vinyl halides, epoxides, cyclic esters and cyclic ethers. The monethylencially unsaturated hydrocarbons include alkyl acrylates, and aryl acrylates. The cyclic ester includes maleic anhydride.

Polymer blends containing COCs have also been found to be suitable for fabricating syringe bodies 1. Suitable two-component blends of the present invention include as a first component a COC in an amount from about 1% to about 99% by weight of the blend, more preferably from about 30% to about 99%, and most preferably from about 35% to about 99% percent by weight of the blend, or any combination or subcombination or ranges therein. In a preferred form of the invention the first component has a glass transition temperature of from about 70° C. to about 130° C. and more preferably from about 70-110° C.

The blends firther include a second component in an amount by weight of the blend of about 99% to about 1%, more preferably from about 70% to about 1% and most preferably from about 65% to about 1%. The second component is selected from the group consisting of homopolymers and copolymers of ethylene, propylene, butene, hexene, octene, nonene, decene and styrene. In a preferred form of the invention the second component is an ethylene and α-olefin copolymer where the α-olefin has from 3-10 carbons, and more preferably from 4-8 carbons. Most preferably the ethylene and α-olefin copolymers are obtained using a metallocene catalyst or a single site catalyst. Suitable catalyst systems, among others, are those disclosed in U.S. Pat. Nos. 5,783,638 and 5,272,236. Suitable ethylene and α-olefin copolymers include those sold by Dow Chemical Company under the AFFINITY and ENGAGE tradenames, those sold by Exxon under the EXACT tradename and those sold by Phillips Chemical Company under the tradename MARLEX.

Suitable three-component blends include as a third component a COC selected from those COCs described above and different from the first component. In a preferred form of the invention the second COC will have a glass transition temperature of higher than about 120° C. when the first COC has a glass transition temperature lower than about 120° C. In a preferred form of the invention, the third component is present in an amount by weight of from about 10-90% by weight of the blend and the first and second components should be present in a ratio of from about 2:1 to about 1:2 respectively of the first component to the second component. about 70-100° C.

In a preferred three-component blend, a second norbornene and ethylene copolymer is added to the two component norbomene-ethylene/ethylene 4-8 carbon α-olefin blend. The second norbornene ethylene copolymer should have a norbornene monomer content of 30 mole percent or greater and more preferably from about 35-75 mole percent and a glass transition temperature of higher than 120° C. when the first component has a glass transition temperature of lower than 120° C.

II. Plunger Seal, Vial Stoppers and Other Elastomeric Components

The plunger seal 6b, vial stopper or other elastomeric component used in conjunction with the COCs set forth above are fabricated from a polymeric material and more preferably a polymeric material that will not generate unacceptable levels of halogens after processing, filling with sterile water for injection, sterilization and storage. More particularly, a syringe body or vial made from one of the COCs set forth above having been filled with 1 ml of sterile water for injection and stoppered with a plunger arm 6a having an elastomeric plunger seal 6b (or other type stopper or closure suitable for the corresponding flowable materials container) will generate less than about 4 ppm of chlorides after three months of storage, more preferably less than about 3 ppm and most preferably less than about 2 ppm of chlorides. In a preferred form of the invention the plunger seal 6b is essentially latex-free and even more preferably 100% latex-free.

In an even more preferred form of the invention the plunger seal 6b and COC body 1 shall meet all limitations set by the United States Pharmocopoeia (Monograph No. 24, effective as of filing this patent application) for sterile water for injection. The USP for sterile water for injection is incorporated herein by reference and made a part hereof. In particular, USP sterile water for injection specifies the following limitations on concentrations: pH shall be from 5.0-7.0, ammonia less than 0.3 mg/ml, chlorides less than 0.5 mg/ml and oxidizable substances less than 0.2 mmol. The USP further specifies the absence of the following components when measured in accordance with the USP: carbon dioxide, sulfates and calcium ions.

Suitable polymeric materials for elastomeric components include synthetic rubbers including styrene-butadiene copolymer, acrylonitrile-butadiene copolymer, neoprene, butyl rubber, polysulfide elastomer, urethane rubbers, stereo rubbers, ethylene-propylene elastomers. In a preferred form of the invention, the elastomeric component is a halogenated butyl rubber and more preferably a chlorobutyl-based elastomner. A presently preferred chlorobutyl-based elastomeric formulation are sold by Stelmi under the trade name ULTRAPURE 6900 and 6901.

It has been further observed that the USP requirements for sterile water for injection are met when the containers of the present invention are prepared using the following methods.

III. Method

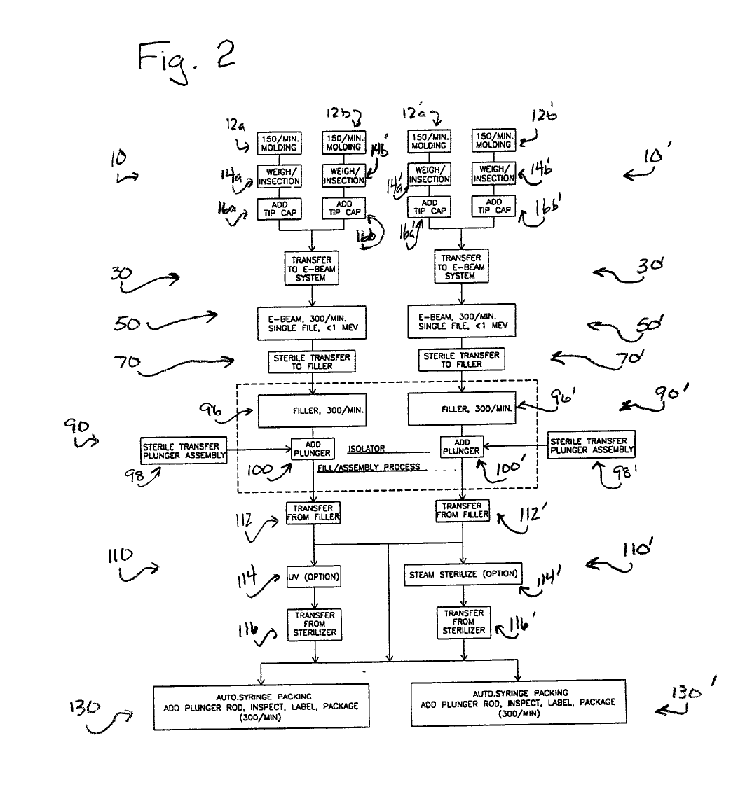

Referring to FIGS. 2 through 4, embodiments of the method of the present invention are illustrated in flowchart format. These embodiments generally comprise the steps of producing a plurality of syringe bodies 10, transferring the syringe bodies to a sterilization station 30, sterilizing the syringe bodies 50, transferring the syringe bodies to a sterile environment 70, processing the syringe bodies within the sterile environment 90, transferring the syringe bodies to a packaging station 110, and packaging the syringe bodies 130.

The methods of producing the sterile prefilled syringe bodies as disclosed herein do not require human intervention. Thus, contamination from human contact is eliminated. To maximize manufacturing of the sterile prefilled syringe bodies dual first and second manufacturing lines may be operated. The second lines are designated by prime reference numerals.

Referring specifically to FIG. 2, the producing the syringe bodies step 10 of this embodiment includes continuously producing a plurality of syringe bodies 12a and 12b. Preferably, the syringe bodies are injection molded from a COC defined above. Typically, the syringe bodies can be molded at a rate of 150 units per minute. Thus, in order to satisfy faster downline subprocesses, two separate 150 unit per minute molding stations 12a and 12b are provided. Once the syringe bodies are molded, they are transferred to a quality control station 14aand 14b where the syringe bodies are inspected and weighed. Syringe bodies which satisfy a predetermined specification are transferred to a tip cap station 16a and 16b where tip caps are added to each syringe body to effectively seal and close the tip end of the syringe body. Next, the interior of the syringe bodies are lubricated, preferably with silicone. The siliconizing can be carried out prior to the tip caps being added without departing from the spirit of the invention.

During the transferring the syringe bodies to a sterilization station step 30, the syringe bodies are transported along a conveyor to a sterilization station. This differs from typical manufacturing methods wherein the syringe bodies are produced at a separate location, placed in nests, trays or tubs, wrapped, transported fro sterilization, sterilized, then transported to manufacturing location where the tubs are unwrapped into an aseptic filling area, filled, and packaged.

The sterilization of the syringe bodies is carried out during the sterilizing the syringe bodies step 50. The sterilization station may include a terminal process performed within an autoclave or an irradiation process. If performed in an autoclave, the sterilization medium is typically steam. Gamma radiation is typically provided to sterilize the syringe bodies through irradiation. In the methods of the present invention, however, electron beam (e-beam) irradiation is preferably provided to sterilize the syringe bodies. Biosterile of Fort Wayne, Indiana supplies an electron accelerator which is capable of sterilizing the syringe bodies. The electron accelerator is sold under the tradename SB5000-4. E-beam irradiation is preferable to steam because irradiation sterilization is faster; it saves manufacturing space; and steam creates waste and causes a material handling problem. E-beam irradiation is preferable over gamma radiation because e-beam irradiation is less damaging to the syringe bodies and it is faster. With e-beam irradiation, there is less coloration of the polymeric material; thus, the clinician's ability to inspect the syringe body and its contents is improved.

The e-beam dose delivered to the syringe bodies is preferably in the range of 10-50 kGy, or any range or combination of ranges therein, and more preferably 25 kGy at approximately 1 MeV to 10 MeV, or any range or combination of ranges therein, but preferably less than or equal to 1 MeV. In studies of the effect e-beam irradiation has on final pH of the medical solutions within the prefilled syringe bodies (which will be described in more detail below), some syringe bodies were given doses greater than 40 kGy.

The dosage may be delivered by a single beam; however, to deliver a uniform dosage to the syringe bodies, a dual beam system is preferred. The dual e-beam system minimizes dosage variation across the syringe bodies. Accordingly, it is further preferred to have an e-beam source located on opposing sides of the conveyor.

Once individual syringe bodies are sterilized, they are sterile transferred to a sterile environment 70 to maintain the sterility of the syringe bodies. The sterile environment is generally a presterilized enclosure in which sterile operations take place under sterile conditions, such as an enclosed isolator, class 100 environment, or other sterile environment. The e-beam sterilization station generates a curtain or field of electrons which provides a sterile ambient atmosphere prior to the syringe bodies entering an adjacent, enclosed, sterile environment or isolator. This is advantageous because the syringe bodies do not need to be wrapped or otherwise sealed to remain sterilized as they are transferred to the sterile environment. In other words, the syringe bodies enter the sterilization station and remain unwrapped and sterilized as they are transferred through the curtain of electrons to the sterile environment. Thus, less handling is required; there is less paper and/or wrapping waste; and it allows the process to proceed continuously because there is no delay for wrapping and unwrapping of the syringe bodies.

The next step, processing the syringe bodies within the sterile environment 90, includes at least three sub-steps, namely filling the syringe bodies with a sterile medical solution 96, transferring a sterile plunger for each syringe body into the sterile environment 98, and adding a plunger to an open end of each syringe body 100. The medical solution is generally introduced by a filler unit provided by Inova GmbH of Schvwabisch Hall, Germany. The medical solution is introduced into the syringe bodies via the open end of the syringe bodies which is opposite the tip capped end, although the medical solution can also be introduced through the tip end without departing from the spirit of the invention.

The plungers are sterilized prior to being transferred into the isolator 98 and may be sterilized in any conventional manner but are preferably processed through the e-beam unit. Once filled with the medical solution, the step of inserting a plunger into the open end of each syringe body 100 is carried out. Once inserted within the open end of the syringe body, the plunger forms a seal with an inner sidewall of the syringe body wherein the medical solution is sealed within the syringe body. The inner sidewall of the syringe bodies have been previously siliconized so that the inner sidewall of the syringe bodies are lubricated, and the plungers will not become fused or adhered to the inner sidewalls. The plungers are automatically added to the syringe bodies as part of the Inova filler process.

The material used to produce the plungers must be compatible with the process. If a material oxidizes as a result of the e-beam irradiation, the oxidizing substances may leach into the contents of the syringe body. Therefore, the stopper is preferably from an elastomeric material such as chlorobutyl rubber, such as Stelmi 6901.

The next step is transferring the syringe bodies to the packaging station 110 from the isolator. In this embodiment, syringe bodies are typically transferred along conveyor; however, any transfer mechanism, such as a manual procedure, a sequent loaders via transfer tubs, or the like, can be used without departing from the spirit of the invention.

This transfer step 110 includes the step of transferring the syringe bodies from the isolator 112 and may optionally include a post-fill sterilization step 114. In this optional sterilization step 114, the syringe bodies and the contents thereof are sterilized either by ultraviolet radiation or steam. The ultraviolet sterilization is performed in-line and takes seconds. Any number of ultraviolet techniques may be employed, such as UV-C (254 nm), medium pressure UV, or pulsed UV. Steam sterilization is performed off-line in an autoclave and generally takes hours.

Following the optional post-fill sterilization step, the syringe bodies are transferred from the optional sterilization station to the packaging station 116. During the packaging station step 130, a plunger rod is fixedly attached to the plunger, and the finished syringes are inspected, labeled, and packaged for shipment to an end user. It is contemplated that no human intervention is required to inspect, label, and package the syringe bodies.

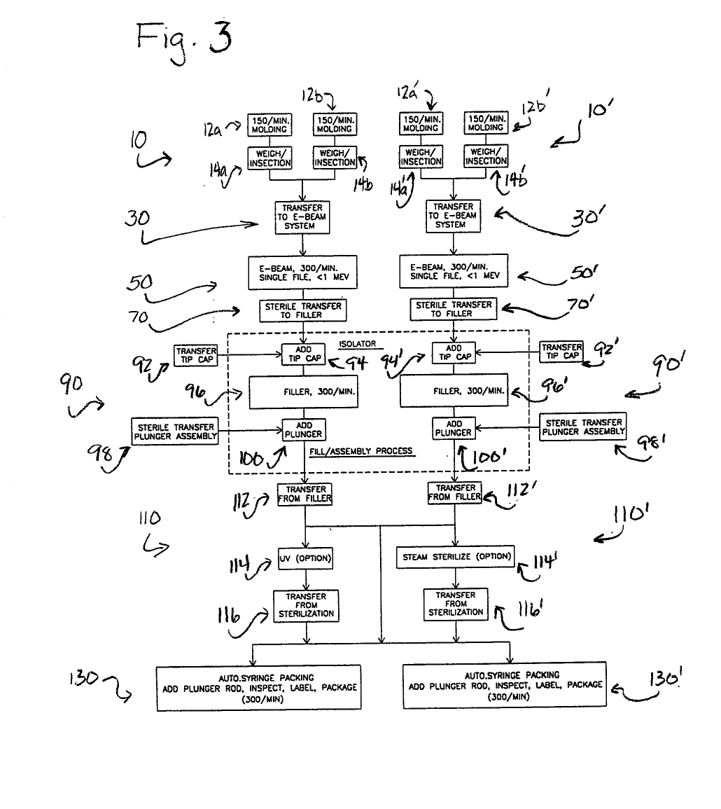

Referring to FIG. 3, a second method of the present invention is illustrated. This method is similar to the first method and also comprises the steps of producing a plurality of syringe bodies 10, transferring the syringe bodies to sterilization station 30, sterilizing the syringe bodies 50, sterile transferring the syringe bodies to a sterile environment 70, processing the syringe bodies within the sterile environment 90, transferring the syringe bodies to a packaging station 110, and packaging the syringe bodies 130.

In this embodiment, the producing the syringe bodies step 10 does not include the sub-step of adding a tip cap to each molded syringe body. Rather, the tip caps are added to the syringe bodies subsequent to sterilization.

Here, the processing the syringe bodies within the sterile environment 90 step at least includes the sub-steps of transferring a sterilized tip cap for each syringe body into the sterilized environment 92, adding a tip cap to an open tip of each syringe body 94, filling the syringe bodies with a medical solution 96, transferring a sterile plunger for each syringe body into the sterile environment 98, and adding the plunger to an open end of a syringe body 100.

The tip caps are sterilized prior to being sterile transferred into the isolator 92 and may be sterilized in any conventional manner but are preferably processed through the e-beam unit or, alternatively, through a separate dedicated e-beam unit. The plungers are processed in a similar manner. The tip caps are preferably added to the open tips of the syringe bodies 94 prior to the syringe bodies being filled with the medical solution 96, and the plungers are preferably added after the syringe bodies have been filled. However, the plungers may be added to the syringe bodies prior to the filling step and the tip caps added to the syringe bodies subsequent to the filling step without departing from the spirit of the invention.

The remaining steps of this embodiment are identical to the first embodiment.

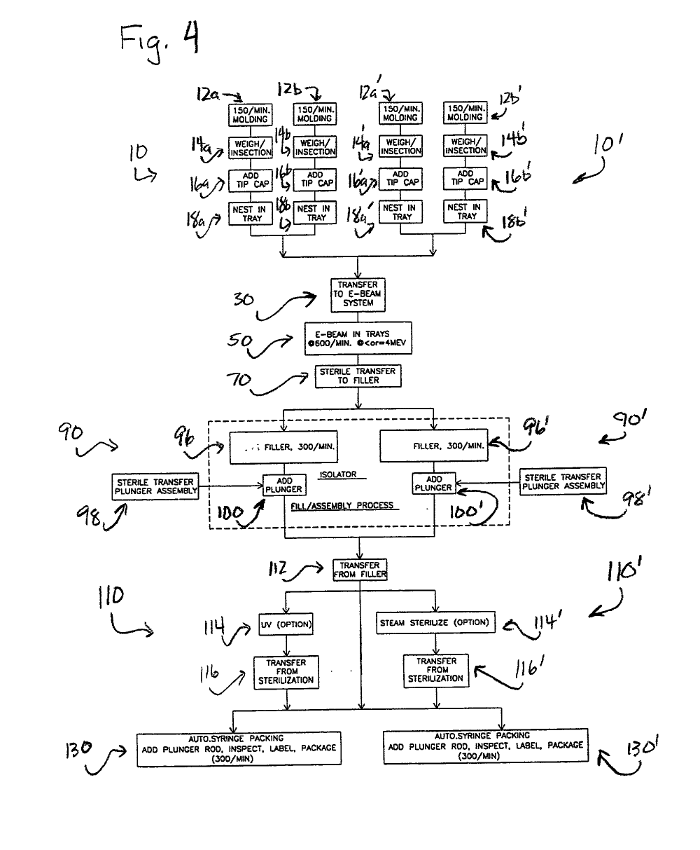

Referring to FIG. 4, a third, preferred embodiment of the method of the present invention is illustrated. In this embodiment, syringe bodies are molded and placed in a transfer tray prior to being transferred to the remaining steps. Thus, rather than a line of syringe bodies being processed through the manufacturing process, a plurality of syringe bodies are transported in a transfer tray through the manufacturing process.

Like the first and second embodiments, this embodiment includes the steps of producing a plurality of syringe bodies 10, transferring the syringe bodies to a sterilization station 30, sterilizing the syringe bodies 50, sterile transferring the syringe bodies to a sterile environment 70, processing the syringe bodies within the sterile environment 90, transferring the syringe bodies to a packaging station 110, and packaging the syringe bodies 130.

Referring specifically to FIG. 4, the producing the syringe bodies step 10 of this embodiment includes continuously producing a plurality of syringe bodies 12a and 12b. Once the syringe bodies are molded, they are transferred to a quality control station 14a and 14b where the syringe bodies are inspected and weighed. Syringe bodies which satisfy a predetermined specification are transferred to a tip cap station 16a and 16b where tip caps are added to each syringe body to effectively seal and close one end of the syringe body. Next, the interior of the syringe bodies are siliconized for lubrication and inserted into a nest located with a transfer tray or tub 18a and 18b. The syringe bodies can be siliconized prior to addition of the tip caps without departing from the spirit of the invention.

During the transferring the syringe bodies to a sterilization station step 30, the syringe bodies are transported within the nested transfer tray along a conveyor to a sterilization station. The sterilization of the syringe bodies is carried out during the sterilizing the syringe body step 50. Again, the sterilization station preferably includes e-beam irradiation. Here, however, the e-beam dose delivered to the syringe bodies must be modified to take into account the increased mass of the plurality of syringe bodies along with the nested transfer tray. Accordingly, the dose of sterilizing irradiation is preferably in the range of 10 to 50 kGy, 20 to 40 kGy, 15 to 25 kGy, or any range or combination of ranges therein, and more preferably 25 kGy at approximately 1 MeV to 10 Mev, more preferably less than or equal to 5 MeV, or any range or combination of ranges therein.

The remaining steps of this embodiment are identical to the first embodiment with the exception that syringe bodies are processed within the nested transfer trays or tubs rather than along the conveyor.

Generally, the sterilized prefilled syringes described herein are filled with a parenteral solution, preferably sterile water for injection. It is important that the pH of the sterile water for injection be controlled and kept within certain upper and lower limits. One advantage of the methods disclosed herein is the tight control of the pH of the water for injection which resulted from using a plastic syringe body sterilized by e-beam irradiation shortly before filling the syringe bodies with sterile water for injection.

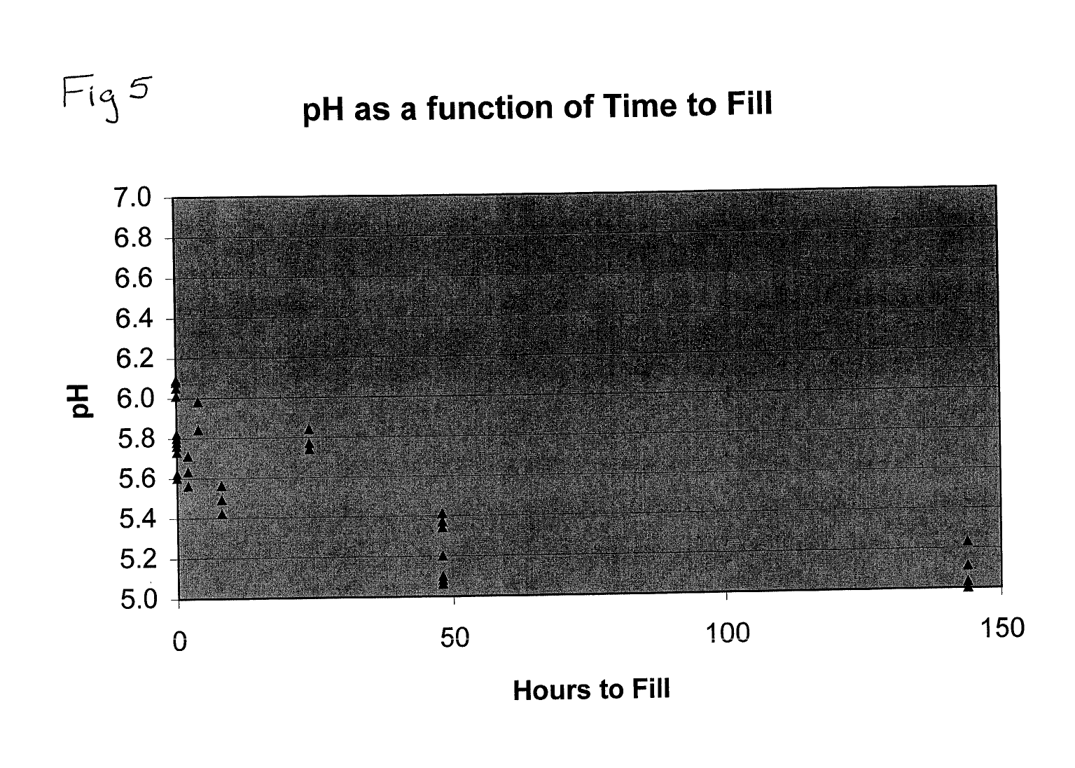

Referring to FIG. 5, the plot illustrates the trend in pH over days to fill. Namely, the pH tends to decrease over time. The following example illustrates an advantage of the present invention; i.e. that sterilization of plastic syringe bodies with e-beam irradiation improved the stability of the solution pH of the sterile water for injection held in the syringe bodies over equivalent gamma irradiation of the syringe bodies.

Syringe bodies were irradiated and aseptically filled within 5 days of e-beam irradiation sterilization. After 3 months in storage at 40 degrees Celsius, 1 mL syringe bodies filled with 1 mL of water which had been sterilized using gamma irradiation (>40 kGy) had a solution pH of 4.71. Meanwhile, syringe bodies stored for 3 months at 40 degrees Celsius which had been sterilized using e-beam irradiation (>40 kGy) had a solution pH of 5.25. Thus, the pH of the sterile water for injection remained within the USP limits of 5.0-7.0 over this time period only for the e-beam irradiated plastic syringe bodies.

Lower doses of e-beam irradiation also maintained the solution pH of water-filled plastic syringes more effectively. Plastic syringe bodies irradiated with doses of e-beam from 20-40 kGy were filled with water within 5 days of sterilization and evaluated after storage. After 2 days storage at 70 degrees Celsius, which appears to approximate at least 2 years storage at 25 degrees Celsius, solution pH remained within USP limits and varied with e-beam dose. The pH of solution was 6.02 at 20 kGy, 5.43 at 30 kGy, and 5.15 at 40 kGy. After 3 months storage at 40 degrees Celsius, 1 mL water-filled syringe bodies yielded pH values of 5.53 at 20 kGy and 5.25 at 40 kGy e-beam irradiation.

The process of filling syringe bodies immediately (within 15 minutes of irradiation) after e-beam irradiation sterilization has been identified as a factor in maintaining the pH of sterile water for injection in small syringe volumes. Plastic syringe bodies were sterilized with e-beam irradiation at 25 kGy and filled with water at various time intervals after irradiation. The syringe bodies were then stored separately for 2 days at ambient temperature and 2 days at 70 degrees Celsius. The solution pH was tested after storage. The results indicated that the immediately filled syringe bodies had substantially higher solution pH than those filled 2 and 6 days after irradiation.

The study was repeated and the results were confirmed with both e-beam and gamma irradiated plungers; thus, predicting that product shelf-life for small volume sterile water for injection filled polymeric syringe bodies may be extended with respect to solution pH by filling the e-beam irradiated polymeric syringe bodies immediately; i.e. within 15 minutes after receiving the e-beam irradiation. It is believed that immediate filling quenches the free radicals formed on the surface of the syringe bodies during irradiation especially when the syringe bodies are produced from a material where ionizing radiation causes the formation of free radicals that could lead to pH changes in the parenteral solution. If a material oxidizes as a result of the e-beam irradiation, the oxidized substances may leach into the contents of the syringe over time. Also, hydrogen peroxide levels of the water have been measured and shown to be quite low (<50 ppb). Therefore, by reducing the pH change caused by the plastic syringe body, the shelf-life of the product is extended.

The following table summarizes the results of the study:

TABLE 1
Immediate Fill of SWFI after E-Beam
Processing of Plastic Syringe Bodies

| | | Ambient | Two Days |
|---|---|---|---|
| | Fill Timing | Control | 70° C. Storage |
| E-beam Irradiated | Filled Immediately | 5.97 | 5.66 |
| (25kGy) Plastic | with 1 mL | | |
| Syringe Bodies with | Filled Immediately | 5.70 | 5.54 |
| E-beam Irradiated | with 10 mL | | |
| (25kGy) | Filled with 10 mL 6 | 5.56 | 5.15 |
| Elastomeric | Days Post-Irradiation | | |
| Plungers | | | |
| E-beam Irradiated 1 | Filled Immediately | 6.09 | 5.77 |
| mL (25kGy) Plastic | Filled 2 Days Post- | 5.78 | 5.08 |
| Syringe Bodies with | Irradiation | | |
| E-beam Irradiated | Filled 6 Days Post- | 5.88 | 5.12 |
| (25kGy) | Irradiation | | |
| Elastomeric | | | |
| Plungers | | | |
| E-beam Irradiated 1 | Filled Immediately | 6.13 | 6.05 |
| mL (25kGY) Plastic | Filled 2 Days Post- | 5.76 | 5.12 |
| Syringe Bodies with | Irradiation | | |
| Gamma Irradiated | Filled 6 Days Post- | 6.00 | 5.02 |
| (25kGy) | Irradiation | | |
| Elastomeric | | | |
| Plungers | | | |

It will be understood that the invention may be embodied in other specific forms without departing from the spirit or central characteristics thereof. The present embodiments, therefore, are to be considered in all respects as illustrative and not restrictive, and the invention is not to be limited to the details given herein.

What is claimed is:

1. An apparatus for producing a sterilized, prefilled container comprising:
a sterilizing station for sterilizing a container;
a sterile environment comprising an opening for receiving a sterilized container;
a sterile ambient atmospheric condition adjacent the opening;
a transport mechanism for transferring a sterilized container from the sterilizing station to the sterile environment wherein the sterilized container is exposed to the sterile ambient atmospheric condition;
a source of a sterile fluid substance; and
a filler for introducing the sterile fluid substance into a sterilized container while the sterilized container is within the sterile environment.

2. The apparatus of claim 1 wherein the sterilizing station comprises a source of electron beam irradiation.

3. The apparatus of claim 2 further comprising a field of electrons produced by the source of electron beam irradiation wherein the field of electrons sterilizes the container and provides at least a portion of the sterile ambient atmospheric condition.

4. The apparatus of claim 2 wherein the source of electron beam irradiation delivers a dose of 10 kGy to 50 kGy.

5. The apparatus of claim 2 wherein the source of electron beam irradiation delivers a dose of 20 kGy to 40 kGy.

6. The apparatus of claim 2 wherein the source of electron beam irradiation delivers a dose of 15 kGy to 25 kGy.

7. The apparatus of claim 2 wherein the source of electron beam irradiation delivers a dose of 25 kGy.

8. The apparatus of claim 4 wherein the dose of electron beam irradiation is delivered at 1 to 10 MeV.

9. The apparatus of claim 4 wherein the dose of electron beam irradiation is delivered at less than 1 MeV.

10. An apparatus for continuous in-line production of a plurality of sterilized, prefilled containers comprising:
a sterilization station comprising a means for continuously sterilizing a plurality of containers;
a sterile isolator comprising an opening for continuously receiving a plurality of containers;
a sterile ambient atmospheric condition adjacent the opening;
a transport mechanism for continuously transferring a plurality of sterilized containers from the sterilizing station to the sterile isolator wherein the plurality of sterilized containers are exposed to the sterile ambient atmospheric condition;
a source of a sterile fluid substance; and
a filler for introducing the sterile fluid substance into a plurality of sterilized containers while the plurality of sterilized containers are within the sterile isolator.

11. The apparatus of claim 10 wherein the means for continuously sterilizing a plurality of containers comprises a source of electron beam irradiation.

12. The apparatus of claim 11 further comprising a field of electrons produced by the source of electron beam irradiation wherein the field of electrons sterilizes the plurality of containers and provides at least a portion of the sterile ambient atmospheric condition.

13. The apparatus of claim 11 wherein the source of electron beam irradiation delivers a dose of 10 kGy to 50 kGy.

14. The apparatus of claim 11 wherein the source of electron beam irradiation delivers a dose of 20 kGy to 40 kGy.

15. The apparatus of claim 11 wherein the source of electron beam irradiation delivers a dose of 15 kGy to 25 kGy.

16. The apparatus of claim 11 wherein the source of electron beam irradiation delivers a dose of 25 kGy.

17. The apparatus of claim 13 wherein the dose of electron beam irradiation is delivered at 1 to 10 MeV.

18. The apparatus of claim 13 wherein the dose of electron beam irradiation is delivered at less than 1 MeV.

19. An apparatus for producing sterilized, prefilled syringe bodies comprising:
a sterilizing station for sterilizing a syringe body;
a sterile isolator comprising an opening for receiving a sterilized syringe body;
a sterile ambient atmospheric condition adjacent the opening;
a transport mechanism for transferring a sterilized syringe body from the sterilizing station to the sterile isolator wherein the sterilized syringe body is exposed to the sterile ambient atmospheric condition;
a source of a sterile fluid substance; and
a filler for introducing the sterile fluid substance into a sterilized syringe body while the sterilized syringe body is within the sterile isolator.

20. An apparatus for continuous in-line production of a plurality of sterilized, prefilled syringe bodies comprising:
a sterilization station comprising a means for continuously sterilizing a plurality of syringe bodies;
a sterile isolator comprising an opening for continuously receiving a plurality of syringe bodies;
a sterile ambient atmospheric condition adjacent the opening;
a transport mechanism for continuously transferring a plurality of sterilized syringe bodies from the sterilizing station to the sterile isolator wherein the plurality of sterilized syringe bodies are exposed to the sterile ambient atmospheric condition;
a source of a sterile fluid substance; and
a filler for introducing the sterile fluid substance into a plurality of sterilized syringe bodies while the plurality of sterilized syringe bodies are within the sterile isolator.

21. The apparatus of claim 20 further comprising a transfer holder adapted for receiving a plurality of syringe bodies wherein the transfer holder is sterilized at the same time the plurality of syringe bodies are sterilized by the sterilization station.

22. The apparatus of claim 20 wherein the means for continuously sterilizing a plurality of containers comprises a source of electron beam irradiation.

23. The apparatus of claim 22 further comprising a field of electrons produced by the source of electron beam irradiation wherein the field of electrons sterilizes the plurality of containers and provides at least a portion of the sterile ambient atmospheric condition.

24. The apparatus of claim 22 wherein the source of electron beam irradiation delivers a dose of 10 kGy to 50 kGy.

25. The apparatus of claim 22 wherein the source of electron beam irradiation delivers a dose of 20 kGy to 40 kGy.

26. The apparatus of claim 22 wherein the source of electron beam irradiation delivers a dose of 15 kGy to 25 kGy.

27. The apparatus of claim 22 wherein the source of electron beam irradiation delivers a dose of 25 kGy.

28. The apparatus of claim 24 wherein the dose of electron beam irradiation is delivered at 1 to 10 MeV.

29. The apparatus of claim 24 wherein the dose of electron beam irradiation is delivered at less than 1 MeV.

30. An apparatus for directly receiving a molded, polymeric container from a container forming process and producing a sterilized, prefilled container comprising:
a sterilization station comprising a means for sterilizing a container;
a means for transporting a container from a container forming process to the sterilization station;
a sterile isolator comprising an opening for receiving a sterilized container;
a sterile ambient atmospheric condition adjacent the opening;
a transport mechanism for transferring a sterilized container from the sterilizing station to the sterile isolator wherein the sterilized container is exposed to the sterile ambient atmospheric condition;
a source of a sterile fluid substance; and
a filler for introducing the sterile fluid substance into a sterilized container while the sterilized container is within the sterile isolator.

31. The apparatus of claim 30 wherein the sterilizing station comprises a source of electron beam irradiation.

32. The apparatus of claim 31 further comprising a field of electrons produced by the source of electron beam irradiation wherein the field of electrons sterilizes the container and provides at least a portion of the sterile ambient atmospheric condition.

33. The apparatus of claim 31 wherein the source of electron beam irradiation delivers a dose of 10 kGy to 50 kGy.

34. The apparatus of claim 31 wherein the source of electron beam irradiation delivers a dose of 20 kGy to 40 kGy.

35. The apparatus of claim 31 wherein the source of electron beam irradiation delivers a dose of 15 kGy to 25 kGy.

36. The apparatus of claim 31 wherein the source of electron beam irradiation delivers a dose of 25 kGy.

37. The apparatus of claim 33 wherein the dose of electron beam irradiation is delivered at 1 to 10 MeV.

38. The apparatus of claim 33 wherein the dose of electron beam irradiation is delivered at less than 1 MeV.

39. A method of in-line, continuous production of sterile prefilled containers, the method comprising the steps of:
providing a container;
sterilizing the container;
providing a sterile environment;
providing a sterile ambient atmospheric condition adjacent the sterile environment;
transferring the sterilized container to the sterile environment while exposing the sterilized container to the sterile ambient atmospheric condition;
providing a source of a medical solution; and
introducing the medical solution into the sterilized container while the sterilized container is within the sterile environment.

40. The method of claim 39 wherein the step of sterilizing the container includes providing a source of electron beam irradiation.

41. The method of claim 40 wherein the step of sterilizing the container includes irradiating the container with a predetermined dose of the electron beam wherein the container is sterilized by the dose of electron beam irradiation and at least a portion of the sterile ambient atmospheric condition includes the predetermined dose of electron beam irradiation.

42. The method of claim 41 wherein the predetermined dose of the electron beam is between 10 kGy and 50 kGy.

43. The method of claim 41 wherein the predetermined dose of the electron beam is 25 kGy.

44. The method of claim 39 further comprising the step of:
sealing the container after the medical solution has been introduced thereto.

45. The method of claim 44 further comprising the step of:
transferring the container from the sterile environment.

46. A sterilized, prefilled container produced according to the method of claim 39.

47. A method of in-line, continuous production of sterile prefilled containers, the method comprising the steps of:
providing a container having no secondary packaging;
sterilizing the container; and
transferring the container to a sterile environment while exposing the container to ambient conditions;
providing a source of a medical solution; and
introducing the medical solution into the sterilized container while the sterilized container is within the sterile environment.

48. The method of claim 47 wherein the step of sterilizing the container includes providing a source of electron beam irradiation.

49. The method of claim 48 wherein the step of sterilizing the container includes irradiating the container with a predetermined dose of the electron beam irradiation wherein the container is sterilized by the dose of electron beam irradiation.

50. The method of claim 49 wherein the predetermined dose of the electron beam irradiation is between 10 kGy and 50 kGy.

51. The method of claim 49 wherein the predetermined dose of the electron beam irradiation is 25 kGy.

52. The method of claim 47 further comprising the step of:
sealing the container after the medical solution has been introduced thereto.

53. The method of claim 50 further comprising the step of:
transferring the container from the sterile environment.

54. A sterilized, prefilled container produced according to the method of claim 47.

55. A method of producing a sterile prefilled container for medical purposes, the method comprising the steps of:
providing a container;
sterilizing the container;
providing a sterile environment;
transferring the sterilized container to the sterile environment while exposing the container to ambient conditions;
providing a fluid substance;
introducing the fluid substance into the container while the container is within the sterile environment; and
sealing the fluid substance within the container while the container is within the sterile environment.

56. The method of claim 55 wherein no human contact with the container is required.

57. The method of claim 55 wherein the sterilizing of the container step includes providing a source of electron beam irradiation and irradiating the container with a predetermined dose of the electron beam irradiation.

58. The method of claim 57 wherein the predetermined dose of the electron beam irradiation is between 10 kGy and 50 kGy.

59. The method of claim 57 wherein the predetermined dose of the electron beam irradiation is 25 kGy.

60. The method of claim 55 wherein the introducing the fluid substance into the container while the container is within the sterile environment step is performed within six days of the sterilizing the container step.

61. The method of claim 60 wherein the fluid substance is a sterile water for injection.

62. The method of claim 61 wherein the sterile water for injection has a pH of solution between 5.0 and 7.0.

63. The method of claim of claim 62 further comprising the steps of:
transferring the container from the sterile environment;
storing the container for a predetermined period of time; and
maintaining a pH of solution of the sterile water for injection within a range of 5.0-7.0.

64. The method of claim 60 wherein the introducing the fluid substance into the container while the container is within the sterile environment step is performed within fifteen minutes after the sterilizing the container step.

65. The method of claim 55 wherein the providing the container step includes forming the container from a polymeric resin.

66. The method of claim 65 wherein the providing container step includes weighing and inspecting the container subsequent to forming the container.

67. The method of claim 66 wherein the forming the container from a polymeric resin includes injection molding the container.

68. The method of claim 65 wherein the polymeric resin is a cyclic olefin copolymer.

69. The method of claim 55 wherein the container is a syringe body.

70. The method of claim 69 further comprising the steps of providing a tip cap for the syringe body and fixing the tip cab to an open tip end of the syringe body.

71. The method of claim 70 further comprising the steps of transferring a sterilized plunger into the sterile environment and inserting the plunger into an open end of the sterile syringe body subsequent to the introducing the fluid substance into the syringe body while the syringe body is within the sterile environment step wherein the the fluid substance is sealed within the syringe body.

72. The method of claim 71 further comprising the step of fixing a plunger rod to the plunger.

73. The method of claim 55 further comprising the steps of transferring the sterilized container from the sterile environment and resterilizing the container subsequent to filling.

74. The method of claim 73 further comprising the steps of labeling the container and packaging the container for delivery to an end user.

75. A prefilled, sterilized container produced according to the method of claim 55.

76. A method of in-line, continuous production of sterile prefilled syringe bodies for medical purposes, the method comprising the steps of:
forming a plurality of syringe bodies by injection molding;
arranging the plurality of syringe bodies in a predetermined order on a transfer mechanism;
transferring the plurality of syringe bodies along the transfer mechanism to a sterilizing location;
sterilizing the plurality of syringe bodies;
transferring the plurality of sterilized syringe bodies to a sterile environment while maintaining the plurality of syringe bodies in a sterilized condition;
providing a fluid substance within the sterile environment;
introducing the fluid substance into the plurality of syringe bodies while the plurality of syringe bodies are within the sterile environment; and
sealing the fluid substance within the plurality of syringe bodies while the plurality of syringe bodies are within the sterile environment.

77. The method of claim 76 wherein the transfer mechanism includes a conveyor belt.

78. The method of claim 77 wherein no human intervention is required.

79. The method of claim 78 wherein the sterilizing of the plurality of syringe bodies step includes providing a source of electron beam irradiation and irradiating the plurality syringe bodies with a predetermined dose of the electron beam irradiation.

80. The method of claim 79 wherein the predetermined dose of the electron beam irradiation is between 10 kGy and 50 kGy.

81. The method of claim 80 wherein the predetermined dose of the electron beam irradiation is 25 kGy.

82. The method of claim 80 wherein the introducing the fluid substance into the plurality of syringe bodies while the plurality of syringe bodies are within the sterile environment step is performed within six days of the sterilizing the plurality of the syringe bodies step.

83. The method of claim 82 wherein the fluid substance is a sterile water for injection.

84. The method of claim 83 wherein the sterile water for injection has a pH of solution between 5.0 and 7.0.

85. The method of claim of claim 84 further comprising the steps of:
transferring the plurality of syringe bodies from the sterile environment;
storing the plurality of syringe bodies for a predetermined period of time; and
maintaining a pH of solution of the sterile water for injection within a range of 5.0-7.0.

86. The method of claim 80 wherein the introducing the fluid substance into the plurality of syringe bodies while the plurality of syringe bodies are within the sterile environment step is performed immediately after the sterilizing the plurality of syringe bodies step.

87. The method of claim 80 wherein the plurality of syringe bodies are formed from a polymeric resin.

88. The method of claim 87 wherein the polymeric resin is a cyclic olefin copolymer.

89. The method of claim 88 further comprising the step of weighing and inspecting the plurality of syringe bodies subsequent to forming the syringe body.

90. The method of claim 80 further comprising the steps of providing a tip cap for each of the plurality of the syringe bodies and fixing the tip cab to an open tip end of each of the plurality of syringe body.

91. The method of claim 90 further comprising the steps of transferring a plurality of sterilized plungers into the sterile environment and inserting at least on of the plurality of plungers into an open end of each of the plurality of sterile syringe bodies subsequent to the introducing the fluid substance into the plurality of syringe bodies while the plurality of syringe bodies are within the sterile environment step wherein the fluid substance is sealed within the plurality of syringe bodies.

92. The method of claim 91 further comprising the step of fixing a plunger rod to each plunger.

93. The method of claim 80 further comprising the steps of transferring the sterilized plurality of syringe bodies from the sterile environment and resterilizing the plurality of syringe bodies subsequent to filling.

94. The method of claim 93 further comprising the steps of labeling the plurality of syringe bodies and packaging the plurality of syringe bodies for delivery to an end user.

95. A method of continuously producing a plurality of sterile prefilled syringe bodies for medical purposes, the method comprising the steps of:
providing a plurality of syringe bodies;
arranging the plurality of syringe bodies within a transfer tray;
sterilizing the plurality of syringe bodies and the transfer tray substantially simultaneously;
transferring the plurality of sterilized syringe bodies and the sterilized transfer tray to a sterile environment while exposing the plurality of syringe bodies and the transfer tray to a sterile ambient atmospheric condition;
providing a fluid substance; and
introducing the fluid substance into each syringe body individually while the plurality of syringe bodies are within the sterile environment.

96. A method of continuously producing a plurality of sterile prefilled syringe bodies for medical purposes, the method comprising the steps, in sequence of:
providing a syringe body;
sterilizing the syringe body;
transferring the sterilized syringe body to a sterile environment while exposing the syringe body to a sterile ambient atmospheric condition;
providing a fluid substance; and
introducing the fluid substance into the syringe body while the syringe body is within the sterile environment.

97. A method of providing a prefilled polymeric container and controlling a solution pH of a sterile, parenteral solution within the polymeric container, the method comprising the steps of:
providing a container produced from a polymeric material where an ionizing radiation causes the formation of free radicals on the container;
providing a source of ionizing radiation;
sterilizing the polymeric container with a predetermined dose of the ionizing radiation;
providing a source of a parenteral solution;
introducing the parenteral solution into the container within 48 hours of sterilizing the polymeric container with a predetermined dose of the ionizing radiation; and
sealing the parenteral solution within the polymeric container.

98. The method of claim 97 wherein the ionizing radiation is an electron beam, irradiation.

99. The method of claim 98 wherein the predetermined dose of electron beam irradiation is between 10 kGy and 50 kGy.

100. The method of claim 99 wherein the predetermined dose of the electron beam irradiation is 25 kGy.

101. The method of claim 97 wherein the introducing the parenteral solution into the polymeric container is performed within 24 hours of the sterilizing the container step.

102. The method of claim 97 wherein the introducing the parenteral solution into the polymeric container is performed within 15 minutes of the sterilizing the container step.

103. The method of claim 102 wherein the parenteral solution has a pH of solution between 5.0 and 7.0.

104. A prefilled, sterile container produced according to the method of claim 97.
